(19) United States
(12) Patent Application Publication
(10) Pub. No.: US 2008/0004236 A1
(43) Pub. Date: Jan. 3, 2008

(54) High Dose, Short Interval Use of Sulfated Polysaccharides for Treatment of Infections

(76) Inventor: Wayne D. Comper, North Ringwood (AU)

Correspondence Address: JONES DAY, 222 EAST 41ST ST, NEW YORK, NY, 10017, US

(21) Appl. No.: 10/588,443

(22) PCT Filed: Feb. 7, 2005
(86) PCT No.: PCT/IB05/02346

§ 371 Date: Jul. 26, 2007

Related U.S. Application Data
(60) Provisional application No. 60/542,743, filed on Feb. 6, 2004.

(51) Int. Cl. A61K 31/715; A61P 43/00

(52) U.S. Cl. 514/54

(57) ABSTRACT

Methods and compositions for treating or preventing acute or chronic viral infection over a short time interval in mammals with sulfated polysaccharides wherein the polysaccharides have a percent of sulfur with respect to the sugar residue effective to enable maximal interaction of constituent sulfate groups with the microbe which causes the infection and wherein the sulfated polysaccharide is not substantially endocytosed or degraded by cell receptor binding in the mammal and thereby retains antiviral activity in vivo.

### 1. FIELD OF THE INVENTION

This invention relates to doses and dosing regimens useful for treating or preventing infections, particularly viral infections, in mammals using sulfated polysaccharides. More particularly, this invention relates to methods of introducing a high dose of a charged and flexible sulfated polysaccharide into the blood stream, lymphatic system and/or extracellular spaces of a patient for the treatment, prevention or management of acute viral infections, acute episodes of chronic viral infections or chronic viral infections. The doses and dosing regimens are particularly well suited for the treatment of acute infections or acute manifestations of viral infections. The most important aspect of this invention is the high dose and short time interval of administration of the compounds of the invention. The invention is best defined by high dose, short use treatment or prevention. Also included within the scope of the invention are single unit dosage forms suitable for high dosing.

### 2. BACKGROUND OF THE INVENTION

Charged polysaccharides, particularly sulfated polysaccharides, have demonstrated potent antimicrobial activities in vitro. (Baba et al., Antiviral Res 9:335-343, 1988; Ito et al., Antiviral Res. 7(36):1-367, 1987). For example, sulfated polysaccharides such as dextran sulfate, heparin, and pentosan polysulfate have been reported to be potent inhibitors of HIV, paramyxoviruses, cytomegaloviruses, influenza viruses, semlikiviruses (Lütscher-Mattli et al., Arch Virol 130:317-326, 1993) and herpes simplex viruses in vitro (Baba et al., Antimicrob. Agents Chemotherapy 32:1742-45, 1988; Pancheva, Antiviral Chem Chemotherapy 4:189-191, 1993). However, the prior uses of these known compounds have demonstrated disappointingly poor activity in vivo.

Dextran sulfate and heparin were first reported to inhibit HIV replication in vitro by Ito et al., Antiviral Res. 7:36 1-367, 1987, Deringer et al. (U.S. Pat. No. 5,153,181) and Ueno and Kuno, Lancet 2:796-97, 1987. Later, several other sulfated polysaccharides were shown to inhibit HIV replication at concentrations believed to be below their respective cytotoxicity thresholds, e.g., pentosan sulfate (Baba et al., Antiviral Res 9: 335-343, 1988; Biesert et al., Aids 2(6):449-57, 1988), fuciodan (Baba et al, Antiviral Res 9:335-343, 1988), lambda-, kappa- and iota-carrageenan (Baba et al., Antiviral Res 9: 335-343, 1988), lentinan sulfate (Yoshida et al., Biochem. Pharmacol. 37(15):2887-91, 1988), mannan sulfate (Ito et al., Eur. J. Clin. Microbiol. Infect. Dis. 8: 191-193, 1989), dextrin sulfate (Ito et al. Antiviral Chem. Chemother., 2:41-44, 1991), sulfoeveman (Weiler et al., J Gen Virol 71:1957-1963, 1990), and sulfated cyclodextrins (Schols et al., J. Acquired Immune Def. Syndr 4:677-85, 1991.). However, these compounds have all proven ineffective in vivo, and at high concentrations cause thromobocytopenia, central nervous system side effects, hair loss, gastro-intestinal pain, anti-coagulation, and the like (Flexner et al., Antimicrob Agents Chemotherapy 35:2544-2550, 1991; Abrams et al., Annals of Internal Medicine (1989) 110:183-188; Hiebert et al., J. Lab & Clin. Med. 133:161-170 (1999)).

Conventional or commercial dextran sulfate has a percent of sulfation of about 17-22%. It is widely accepted that increasing sulfur content of dextran sulfate increases the anticoagulant activity of the material. (Hirata et al., Biosci. Biotech. Biochem. 58(2):406-407, 1994). Similarly, it is widely accepted that increasing the sulfur content of sulfated polysaccharides increases their in vitro antiviral activity. See, e.g., Witvrouw et al., General Pharmacology 29 (4): 497-512, 1997; Nakashima et al., Jpn. J. Cancer Res. (Gann) 78:1164-68, 1987; and Baba et al., J. AIDS 493-499, 1990. Again, these studies have demonstrated a marked increase in the in vitro activity of sulfated polysaccharides with the increase in sulfation, although the lack of in vivo efficacy remained. Indeed, lack of in vivo efficacy and the in vivo toxicity of compounds with a high degree of sulfation has been an unsolvable problem to date.

Although there have been a limited number of studies of sulfated polysaccharides with lower percents of sulfation for specific uses, these materials have not been characterized with respect to both their molecular weight and their percent of sulfation. Significantly, these materials have been reported to be less active against retroviruses than polysaccharides with 17-22% sulfation, e.g., levels similar to or the same as commercial dextran sulfate. Id. Further, poorly characterized (if characterized at all), low molecular weight preparations have been studied in animals for activity against herpes virus as in EP Application 0 066 379 A2 with limited success. (See also, Pancheva S N. Antiviral Chem Chemotherapy 4:189-191, 1993.)

Considerable effort has been focused on improving the in vivo anti-viral activity of dextran sulfate by increasing its sulfation or modifying the use of conventional material. In one study, given the reported poor absorption of oral dextran sulfate, dextran sulfate was administered to a maximally tolerated dose by continuous infusion to subjects with symptomatic HIV infection for up to 14 days. (Flexner et al., Antimicrob Agents Chemotherapy 35:2544-2550, 1991). Continuous intravenous infusion of dextran sulfate was found to be toxic. The authors concluded that as a result of its toxicity and lack of any demonstration of beneficial effect in vivo, dextran sulfate is unlikely to have a beneficial effect in the treatment of HIV. Id. Indeed, the authors cautioned: “further clinical development of parenteral dextran sulfate as therapy for symptomatic HIV infection is not warranted and could prove to be hazardous. On the basis of the results of this study, caution is advised in the clinical evaluation of other polysulfated polyanions.” (Id. at 2549).

In sum, although commercial dextran sulfate has been previously used in Japan for anticoagulation and hyperlipidemia, it has demonstrated poor activity against HIV in vivo or, dextran sulfate has been reported to have significant toxicity in mammals and HIV patients. (Mathis et al., Antimicrobial Agents & Chemotherapy 2147-2150, 1991; Flexner et al., Id. 2544-2550; Abrams et al., Annals of Internal Medicine 110:183-188 (1989); Hiebert et al., J. Lab & Clin. Med. 133:161-170 (1999)).

There is a need for dosing regimens that can be used to exploit the activity of molecules such as sulfated polysaccharides without toxicity that limits or prevents therapeutic or prophylactic uses.

### 3. SUMMARY OF THE INVENTION

Applicant has previously and unexpectedly discovered that synthetic sulfated polysaccharides having a percent of sulfur between 6 and 13% are effective in vivo against microbial infections, particularly viruses. (See, e.g., Applicant's copending United States Application Publication Nos. 2003/0181416, published Sep. 25, 2003, and 2004/0009953, published Jan. 15, 2004, which are hereby incorporated by reference). This discovery was made despite the accepted belief that commercial dextran sulfate was either ineffective in vivo or toxic in vivo or both.

Applicant has now discovered that despite the toxicity associated with the administration of sulfated polysaccharides at high doses, such doses are effective for the treatment of viral infections, preferably acute viral infections or acute episodes and crisis periods of chronic infection, particularly when administered over short time intervals. The invention encompasses short term administration of the compounds of the invention at high doses wherein the short term high dose is sufficient to treat an acute infection while reducing or avoiding toxicity of a severity, irreversibility or seriousness that would preclude its use as a therapeutic. While central nervous system side effects, hair loss, gastro-intestinal pain, bowel hemorrhaging, listlessness, thrombocytopenia, central nervous system damage, headache, pain, fever, asthenia, chills, malaise, syncope, vasodilatation, nausea, diarrhea, dyspepsia, anorexia, anemia, dizziness, muscle spasm, sinusitis, urticaria, alopecia, anorexia, constipation or anti-coagulation will likely result to some degree upon administration of high doses of sulfated polysaccharides, none of the side effects are unmanageable or permanent if the doses are administered in accordance with this invention, that is most patients recover from these side effects in hours or days after administration. Thus, despite such side effects, the administration of a high dose provides a viable option for patients faced with serious infections, especially acute viral infection or other infections putting patients in a crisis situation.

Furthermore, Applicant has discovered that the treatment or management of chronic viral infections can also be effectively undertaken by the administration of sulfated polysaccharides at high doses, particularly with single or repeated short dosing regimens. Adjustments of the inception and repetition of the doses will, of course, vary with the treatment of acute versus chronic diseases.

In one embodiment, the invention encompasses novel methods using high doses and certain administration regimes for the treatment or management of acute viral infection, chronic viral infection, or acute episodes of chronic viral infections which utilize sulfated polysaccharides, including naturally occurring, non synthetic and commercially available polysaccharides, particularly dextran sulfates. The invention encompasses in a preferred embodiment the use of sulfated polysaccharides, having a percent of sulfur with respect to the simple sugar residue of greater than 2% and less than 25%, more preferably greater than 2% and less than 6%, greater than 6% and less than 13% or greater than 13% and less than 25%, within the methods and compositions of the invention. Preferred sulfated polysaccharides are dextran sulfate and sulfated polysaccharides, having a percent of sulfur with respect to the simple sugar residue of greater than 6% and less than 13%. The sulfated polysaccharides are preferably sulfated dextrans having an α-1,6-glycosidic linkage.

The methods of the present invention are particularly well suited for the treatment of acute viral infection, including, but not limited to severe acute respiratory syndrome (SARS)-associated coronavirus. For example, the methods of the present invention can be administered immediately following demonstration of symptoms or other manifestations of acute infections. Similarly, the methods can be uses following first exposure to, or infection by, a particular virus, such as HIV, to lessen or avoid a more serious infection. Similarly, the methods of the invention can be repeatedly administered over time for the management of chronic infections, including, but not limited to herpesvirus and HIV by administration of high doses of sulfated polysaccharides for short periods of time or during acute episodes or acute crisis periods of chronic infections. Very high doses of the sulfated polysaccharides administered over relatively short periods of time can alleviate the serious consequences of the acute infection. Any toxic side effects of the sulfated polysaccharides of the invention will be short lived and reversible.

In one embodiment, the invention further encompasses the use of sulfated polysaccharides having a molecular weight between 500 and 10,000,000, preferably above 1,000; more preferably above 20,000; most preferably above 40,000, within the methods and compositions. Ranges of 1,000 to 1,000,000; 25,000 to 500,000; and 40,000 to 300,000 are also encompassed by the invention for oral or parenteral use. In another alternative embodiment for topical administration, the sulfated polysaccharide may have a molecular weight of 5,000 to 10,000,000 but preferably higher than 500,000. In an alternative embodiment, the composition has only about 10% variability in the molecular weight and preferably about 5% variation.

In a preferred embodiment the sulfated polysaccharide is dextran sulfate. In addition to dextran sulfate, the sulfated polysaccharide can be cellulose sulfate, dextrin sulfate, cyclodextrin, or one of the other materials found in Table 1 below, preferably wherein the percent of sulfur is within the range of 2% to 25%, more preferably greater than 6% and less than 13%, or greater than 13% and less than 25%, even more preferably greater than 8% and less than 22%, and most preferably greater than 6% and less than 13%. Moreover, substituted polysaccharides such as carboxymethyl substituted or periodated treated sulfated polysaccharides, particularly substituted dextran sulfates such as carboxymethyl substituted dextran sulfate or periodate treated dextran sulfates can be used. In one embodiment, the sulfated polysaccharide is homogenous with respect to molecular weight, percent of sulfation or both.

The viral infections encompassed by the methods of the invention, particularly the specific viruses to be treated and specific sulfated dextrans to be used, are described in detail below.

### 3.1 Definitions

As used herein, the term “patient” or “subject” means an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In certain embodiments, the patient is an infant, child, adolescent, adult or geriatric patient. In addition, the patient includes immunocompromised patients such as HIV positive patients, cancer patients, patients undergoing immunotherapy or chemotherapy.

As used herein, a “therapeutically effective amount” refers to an amount of the sulfated polysaccharide of the invention sufficient to provide a benefit in the treatment or management of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses an amount that improves overall therapy, reduces or avoids symptoms or causes of disease, or enhances the therapeutic efficacy of or synergies with another therapeutic agent.

As used herein, a “high dose” refers to an amount of the sulfated polysaccharide of the invention sufficient to provide a benefit in the treatment or management of viral disease, or to cure or ameliorate the disease, infection or cause thereof, while achieving only certain non-lethal toxicities. In particular, a high dose means an amount sufficient to provide a therapeutic benefit in vivo and is generally at or just below the maximum tolerated dose thereby resulting in temporary or reversible side effects. Used in connection with an amount of a compound of the invention, the term preferably encompasses an amount that improves overall viral load, reduces viral replication or causes of disease, or enhances the therapeutic efficacy of or synergies with another therapeutic agent. High doses utilized in this invention is analogous to those used by oncologists or radiologists in treating tumors where toxicity to healthy cells is tolerated in order to exploit the benefits of the treatment despite toxic side effects.

As used herein, “in combination” refers to the use of more than one prophylactic and/or therapeutic agents simultaneously or sequentially and in a manner that their respective effects are additive or synergistic.

As used herein, the terms “manage”, “managing”, and “management” refer to the slowing or preventing the progression or worsening of the viral infection, reducing the viral load, or preventing the death or serious symptoms or effects associated with viral infection.

As used herein, the terms “treat”, “treating” and “treatment” refer to the eradication or amelioration of the infection itself, causes of the infection, or symptoms associated therewith. In certain embodiments, such terms refer to minimizing the spread or worsening of the infection resulting from the administration of one or more prophylactic or therapeutic agents to a subject with such an infection.

As used herein, the terms “acute”, “acute infection” and “acute viral infection” refer to brief health effects of a viral infection; brief, intense or short term exposure to a virus; a brief period of increased manifestation of a virus or a first or significant exposure to a virus, e.g., infection by, and symptoms of, rhinoviral, coronaviral, poxviral infection.

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compound of the present invention include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

As used herein and unless otherwise indicated, the term “optically pure” or “stereomerically pure” means a composition that comprises one stereoisomer of a compound and is substantially free of other stereoisomers of that compound. For example, a stereomerically pure a compound having one chiral center will be substantially free of the opposite enantiomer of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. Since the compounds of the invention are primarily polysaccharides made of saccharides which can exist in either the D or L forms, the invention encompasses either or both D and L sugars. As such, for example, a stereomerically pure D sugar will be substantially free of the L form. In an alternative embodiment, the use of L forms of sulfated dextrans permits the use of a broader controlled range of sulfation from above 2% to about 30%. Thus, the methods and compositions disclosed herein include in an alternative embodiment the use of such levorotatory sugars or polymers made therefrom.

As used herein, the term “sulfated polysaccharide” means a naturally occurring, non-synthetic, or synthetic sulfated material having more than ten units of simple sugar. Preferably the sulfated polysaccharide is an alpha(1,6) linked polysaccharide, more preferably commercial dextran sulfate and most preferably sulfated polysaccharides having a percent of sulfur between about 6% and about 13%. Ranges of sulfur content are described in more detail below.

As used herein, the term “dextran” means a polysaccharide containing a backbone of D-glucose units linked predominantly α-D(1,6), composed exclusively of α-D-glucopyranosyl units differing only in degree of branching and chain length.

As used herein, the term “dextran sulfate sodium” or “dextran sulfate”, “conventional dextran sulfate”, or “commercial dextran sulfate” unless otherwise qualified means a α-1,6-polyglucose containing approximately 17% sulfur with up to three sulfate groups per glucose molecule of varying molecular weight ranges, e.g., 4,000-500,000 Da.

As used herein, the terms “percent sulfation”, “percent of sulfation”, “percent of sulfate substitution” or “sulfation” means the percent of sulfur by molecular weight with respect to each simple sugar residue within the polysaccharide in question, optionally including a counterion, e.g., molecular weight of sulfation in the composition/total weight. In a preferred embodiment, the percent of sulfur is calculated as the percent of sulfur by molecular weight with respect to the sulfated sugar residue within the polysaccharide in question with sodium as the counterion. The percent of sulfation can be determined by elemental analysis of material which has been dialyzed to remove free sulfur, preferably of moisture/volatile free material dried in vacuo at 60° C. to a constant weight. Other methods of determining percent of sulfation are via moisture content analysis and titration. Sulfation is to be distinguished from “degree of substitution” or “equivalents” which is a measure of the number of sulfate groups per sugar moiety. However, it will be recognized by one of skill in the art that percent sulfation can be converted to a degree of substitution or equivalents and vice versa.

As used herein, the term “co-charged dextran polyanions” is dextran substituted to varying degrees with any combination of carboxymethyl groups, sulfate groups and sulfonate groups.

As used herein, the term “periodate treated anionic polysaccharides” means any anionic polysaccharide that has been treated with periodate to open the sugar ring without depolymerization or to otherwise increase the flexibility of the polysaccharide in order to increase interaction with the virus.

### 4. BRIEF DESCRIPTION OF THE DRAWINGS

FIG. 1 is a schematic flowchart describing the preparation of sulfated dextrans of a specific percent of sulfation and molecular weights.

### 5. DETAILED DESCRIPTION OF THE INVENTION

Contrary to the teachings in the literature, the present invention encompasses the in vivo use of high-doses of sulfated polysaccharides, including those with a range of sulfur content below, above or including 6% to 13% to treat acute viral infections, chronic viral infections, and acute episodes of chronic infections.

The most important aspect of this invention is the high dose and short time interval of administration of the compounds of the invention. The invention is best defined by the use of doses of sulfated polysaccharides that are at, near or below the maximum tolerated doses. Even though such doses will lead to levels of toxicity to healthy cells, that toxicity is temporary, reversible and non-lethal and the benefits against infection will be significant. Avoiding prolonged and continued use of these high doses improves the therapeutic profile of the high dose methods. Of course, repeated use can be as the patient is monitored.

In one embodiment of the invention, the invention encompasses sulfated polysaccharides such as conventional dextran sulfate or variants thereof (e.g., dextran sulfate with a percent of sulfur that differs from the conventional material) that can be used to treat or prevent viral infection. In one embodiment, the sulfated polysaccharide has a percent of sulfation greater than 2% but below 25% range, preferably greater than 2% and less than 6%, greater than 6% and less than 13% or greater than 13% and less than 25%, more preferably greater than 7% and less than 22%, most preferably greater than 13% and less than 18%. The most preferred compositions or methods of the invention utilize sulfated α-1,6-linked polysaccharides or sulfated dextrans having the desired percent of sulfation and/or molecular weight which are flexible and thus useful against a wide variety of viruses. In a most preferred embodiment, the range of percent sulfation is effective to enable maximal interaction of constituent sulfate groups with the virus which causes the infection, and wherein the sulfated polysaccharide is not substantially endocytosed or degraded by cell receptor binding in the mammal, and thereby retains antiviral activity in vivo.

Thus, the present invention encompasses methods for treating or managing acute viral infections, chronic viral infections or acute episodes of chronic viral infections in vivo, with a high dose of a sulfated polysaccharide or a pharmaceutically acceptable salt, hydrate, or stereoisomer thereof, having flexibility in its structure, a controlled degree of sulfation, and optionally homogeneity as to its molecular weight, and low degree of sulfation as compared to conventional dextran sulfate.

The present invention also provides methods for the treatment, or management of acute or chronic viral infection comprising administering to a patient in need thereof a high dose of a sulfated polysaccharide or pharmaceutically acceptable salts, hydrates, or stereoisomers thereof having from greater than 2% to below 25% sulfation.

Without being limited by any particular theory, the Applicant believes that there is a range of charge density for sulfated polysaccharides within which they exhibit anti-viral activity in vitro and retain their anti-viral activity in vivo. In a preferred embodiment of the invention, the sulfated polysaccharides of the invention have a percent of sulfation of greater than 2% and less than 6%, greater than 6% and less than 13% or greater than 13% and less than 25%, preferably greater than about 7% and below 22%, most preferably 2.5%, 3%, 3.5%, 4%, 4.5%, 5%, 5.5%, 6.0%, 6.5%, 7.0%, 7.5%, 8.0%, 8.5%, 9%, 9.5%, 10%, 10.5%, 11%, 11.5%, 12%, 12.2%, 12.5%, 12.8%, 13.5%, 14%, 14.5%, 15%, 15.5%, 16%, 16.5%, 16.8%, 17%, 17.5%, 18%, 18.5%, 19%, 19.5%, 20%, 20.5%, 21%, 21.5%, 22%, 22.5%, 23%, 23.5%, 24% or 24.5% within±1%.

A preferred sulfated polysaccharide used in the methods of the invention is sulfated dextran, or an α-1,6-linked polysaccharide, which has been modified to have the appropriate percent of sulfation. The sulfated dextran of the invention contain less than 25%, and may contain less than 17%, less than 16%, less than about 15%, less than 14%, but more than 13% sulfur. The sulfated dextran of the invention may also contain less than 6%, and may contain less than 5%, less than 3%, less than about 3%, but more than 2% sulfur. In a preferred embodiment, the sulfated dextran variant has a sulfation of less than 17% and greater than 13%.

The invention further encompasses the use of sulfated polysaccharides having a molecular weight between 500 and 10,000,000, preferably above 5,000; more preferably above 25,000; more preferably above 40,000; and most preferably 7,500, 10,000, 12,500, 15,000, 17,500, 20,000, 22,500, 25,000 27,500, 30,000, 32,500, 35,000, 37,500, 40,000, 42,500, 45,000, 47,500; 50,000, 52,500, 55,000, 57,500 or 60,000 within the methods and compositions. Ranges of 5,000 to 10,000,000; 25,000 to 500,000; and 40,000 to 300,000 are also encompassed by the invention for oral or parenteral use. In general, for topical administration, the sulfated polysaccharide may have a molecular weight of 5,000 to 10,000,000 but preferably higher than 500,000. In an alternative embodiment, the composition has only about 10% variability in the molecular weight and preferably about 5% variation.

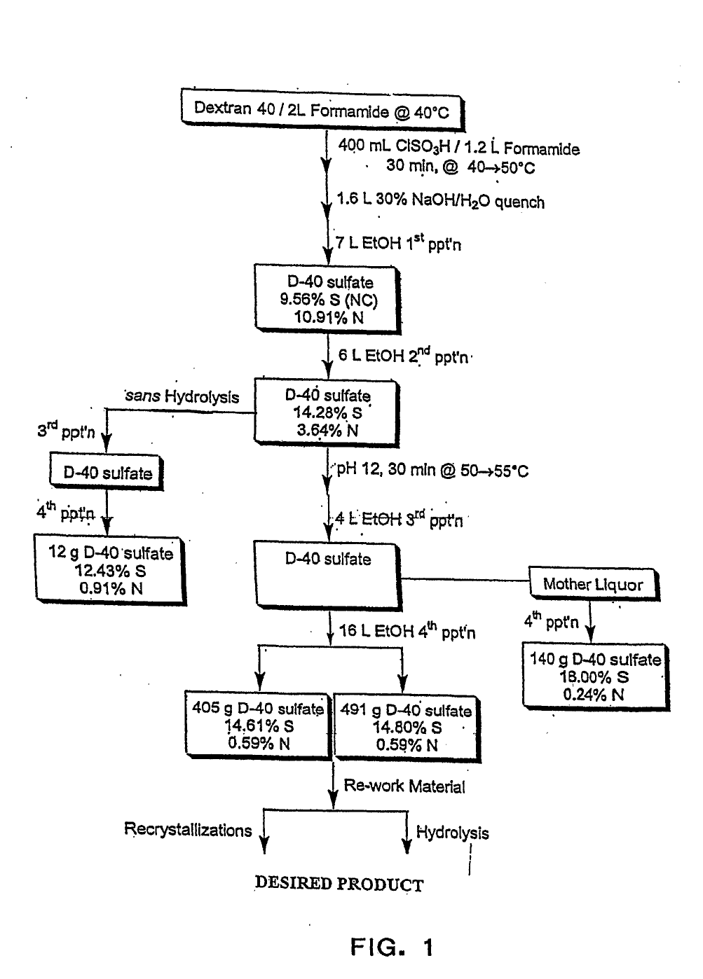

The sulfated polysaccharides of the invention can be naturally occurring, non-synthetic or synthetic. The synthetic sulfated polysaccharides, particularly the sulfated dextrans, can be prepared using known synthetic techniques and reagents. Several methods which are known in the art may be modified so that the proper degree of sulfation is achieved. These methods include those described in FIG. 1. However, as mentioned above, one may control the molecular weight as well as the degree of sulfation. Applicant has synthesized sulfated dextran with controlled sulfur contents and controlled degrees of sulfate substitution so that they are not taken up by cell receptors for highly charged polysaccharides. These polysaccharides exhibit essentially the same high antiviral activity in vivo as they do in vitro and have enhanced stability and longevity in vivo, as they are not readily taken up by cells they are also less toxic. Or more generally, as the sulfur content is decreased, activity and toxicity decrease. Sulfated dextran, with controlled sulfur content is particularly well suited as a viral cell attachment inhibitor because of its unique structure—essentially linear chain composed of an α-1,6-glycosidic linkage which makes it a more flexible polysaccharide—that enables maximal interaction of its constituent sulfate groups with positive charges on proteins of the virus but does not bind significantly to plasma proteins including albumin.

In another alternative embodiment, the invention encompasses the use of homogeneous sulfated polysaccharides. That is to say the sulfated polysaccharides administered in accordance with the methods described herein or utilized in the pharmaceutical compositions and dosage forms exhibit substantially the same percent of sulfation or molecular weight or both.

In a separate embodiment, the invention encompasses a method of treating or preventing a viral infection in a mammal comprising administering to a mammal in need thereof a high dose of a composition comprising a sulfated polysaccharide having a percent of sulfate substitution per glucose residue in the polysaccharide ranging from greater than 2% to less than 25%, wherein the range of percent sulfation is effective to enable maximal interaction of constituent sulfate groups with the virus which causes the infection, and wherein the sulfated polysaccharide is not substantially endocytosed or degraded by cell receptor binding in the mammal, and thereby retains antiviral activity in vivo. Preferably, the sulfated polysaccharide is sulfated dextran; more preferably, the sulfated polysaccharide is commercial dextran sulfate or a sulfated polysaccharide having a percent of sulfur between about 6% and about 13%.

The invention also encompasses the treatment, prevention or management of anti-inflammatory diseases or disorders, interstitial cystisis and anti-arthritic diseases with high doses or high dose regimens. The invention also encompasses the use of the sulfated polysaccharides of the invention as anti-albuminuric agents (albuminuria that occurs in kidney disease) with high doses or high dose regimens.

The invention further encompasses a method of treating or preventing a viral infection in a mammal which comprises administering to a mammal in need thereof an effective amount of a levorotatory sulfated polysaccharide having a percent of sulfation from about 2% to about 25%; preferably greater than 2% and less than 6%, greater than 6% and less than 13% or greater than 13% and less than 25%; more preferably from about 7% to about 17%.

In a further embodiment, the invention encompasses a method of treating or preventing a viral infection in a mammal which comprises administering to a mammal in need thereof of a periodate-treated anionic polysaccharide. Preferably, the periodate-treated anionic polysaccharide is a periodate treated sulfated dextran.

In another embodiment of the invention, the invention encompasses a method of treating or preventing a viral infection in a mammal which comprises administering to a mammal in need of such treatment or prevention a high dose of a co-charged anionic polysaccharide which has a percent of sulfation which enables maximal interaction with the virus and which is not substantially endocytosed or degraded by cell receptor binding in the mammal thereby retaining antiviral in vivo. In a preferred embodiment, the co-charged anionic polysaccharide is co-charged with carboxymethyl groups, sulfonate groups, sulfate groups or mixtures thereof; more preferably the co-charged anionic polysaccharide is co-charged with carboxymethyl groups. In a specific embodiment, the co-charged anionic polysaccharide is carboxymethyl dextran sulfate or carboxymethyl cellulose.

In another embodiment of the invention, and depending on the specific tissue to be treated, additional components; including, but not limited to penetration or absorption enhancers, molecules that target the area of the infection and molecules that reduce the in vivo toxicity of the sulfate polysaccharide; may be used prior to, in conjunction with, or subsequent to treatment with one or more high doses of the sulfated polysaccharides of the invention.

5.1 Viral Infections

Acute viral infections, chronic viral infections and acute episodes of chronic viral infections which can be treated, prevented or managed by the methods of the present invention include, but are not limited to DNA and RNA viruses. The DNA and RNA viruses within the scope of the invention include, but are not limited to double-stranded DNA viruses, single-stranded DNA viruses, DNA reverse transcripting viruses, RNA reverse transcripting viruses, double-stranded RNA viruses, negative-sense single stranded RNA viruses, positive-sense single-stranded RNA viruses, and ambisense RNA viruses. In one specific embodiment, the methods and compositions can be used to treat, prevent or manage infection of non-enveloped viruses, including but not limited to, picornaviruses, caliciviruses, astroviruses, reoviruses, birnaviruses, circoviruses, parvoviruses, papovaviruses, and adenoviruses.

In preferred specific embodiment, the methods and compositions can be used to treat, prevent or manage infection of enveloped viruses, including but not limited to, togaviruses, flaviviruses, rhabdoviruses, filoviruses, paramyxoviruses, orthomyxoviruses, bunyaviruses, arenaviruses, retroviruses, hepadnaviruses, herpesviruses, poxviruses, coronaviruses, iridoviruses, and arteriviruses.

Specific enveloped double-stranded DNA viruses which can be treated, prevented or managed by the methods of the present invention include, but are not limited to, Herpesvirus B virus (Cercopithecus herpesvirus 1), Cowpox virus, Epstein-Barr virus (human herpesvirus 4), Hepatitis B virus, Herpes simplex viruses 1 and 2 (HSV-1 and -2), Human cytomegalovirus (human herpesvirus 5), Human herpesviruses 6A, 6B and 7, Molluscum contagiosum virus, Monkeypox virus, Pseudocowpox virus, Tanapox virus, Vaccinia virus, Varicella-zoster virus, Variola virus (smallpox virus), African swine fever virus, Bovine mamillities virus, Bovine papular stomatitis virus, Chelonoid herpesvirus 1, Cowpox virus, Ectromelia virus (mousepox virus), Equine abortion virus (EHV1), Equine coital exanthema virus (EHV3), Equine rhinopneumonitis virus (EHV4), Fibroma viruses (of rabbits, hares and squirrels), Frog viruses 1-3, 5-24, L2, L4, and L5, Fowlpox virus, Goldfish viruses 1-2, Infectious bovine rhinotracheitis virus, Infectiuos bovine rhinotracheitis virus, Infectious laryngotracheitis virus (fowl), Lymphocystis disease virus (fish), Marek's disease virus (fowl), Movar herpesvirus, Myxoma virus, Orf virus (contagious pustular dermatitis virus), Pseudocowpox virus (milker's nodule virus), Pseudorabies virus, Sheeppox virus, Swinepox virus, Yabapox virus, and Woodchuck hepatitis virus.

Specific non-enveloped double-stranded DNA viruses which can be treated, prevented or managed by the methods of the present invention include, but are not limited to Adenovirus 1-49, Simian adenoviruses 1-27, Bovine adenoviruses 1-9, Porcine adenoviruses 1-4, Ovine adenoviruses 1-6, Equine adenoviruses 1-2, Murine adenoviruses 1-2, BK virus, JC virus, K virus (rabbits), Rabbit kidney vacuolating virus, Papillomaviruses 1-60, Simian virus 12 (SV 12), Simian virus 40 (SV 40), Bovine papillomaviruses 1, 2, and 4, Canine oral papillomavirus, Canine adenovirus 2, equine papillomavirus, ovine papillomavirus, Equine adenoviruses, Fetal rhesus kidney virus, Infectious canine hepatitis virus, Mouse polyoma virus, African green monkey B-lymphotropic polyoma virus, and Shope papillomavirus.

Specific non-enveloped single-stranded DNA viruses which can be treated, prevented or managed by the methods of the present invention include, but are not limited to Parvovirus B-19, RA-1 virus, Aleutian mink disease virus, Canine parvovirus, Mink enteritis virus, Minute virus of mice, Chicken anemia virus, Psittacine beak and feather disease virus, and Porcine circovirus.

Specific non-enveloped single-stranded positive sense RNA viruses which can be treated, prevented or managed by the methods of the present invention include, but are not limited to Coxsackieviruses A 1-21 and A24, Coxsackieviruses B 1-6, Echoviruses 1-7, 9, 11-27 and 29-34, Enteroviruses 68-71, Hepatitis A virus, Hepatitis E virus, Norwalk and similar viruses (such as Southampton, Snow Mountain, Hi., and Taunton viruses), Polioviruses 1-3, Rhinoviruses 1-113, 1A, and 1B, Bovine enteroviruses 1-7, Encephalomyocarditis virus, Feline calicivirus, Foot-and-mouth disease viruses, Mouse poliomyelitis virus (Theiler's virus), Murine encephalomyelitis virus, Porcine enteroviruses 1-8, Bovine enteroviruses 1-7, Simian enteroviruses 1-18, Rabbit hemorrhagic disease virus, Swine vesicular disease virus, Vesicular exanthema viruses 1-12 (swine), Chimpanzee calicivirus (Pan-1), San Miguel sea lion viruses 1-8, European brown hare disease virus, Feline calicivirus, Canine calicivirus, Bovine enteric calicivirus, Porcine enteric calicivirus, Mink calicivirus, Reptile calicivirus, Walrus calicivirus, Fowl calicivirus, Human astroviruses 1-5, Bovine astroviruses 1-2, Ovine astrovirus, Porcine astrovirus, Canine astrovirus, and Duck astrovirus.

Specific enveloped single-stranded positive sense RNA viruses which can be treated, prevented or managed by the methods of the present invention include, but are not limited to Barmah Forest virus, Central European encephalitis virus, Chikungunya virus, Dengue viruses 1-4, Eastern equine encephalitis virus, Hepatitis C virus, Human immunodeficiency viruses 1 and 2, Human T-lymphotropic viruses 1 and 2, Igbo Ora virus, Japanese encephalitis virus, Kyasanur forest virus, Mayaro virus, Murray Valley encephalitis virus, O'nyong-nyong virus, Omsk hemorrhagic fever virus, Rocio virus, Ross River virus, Rubella virus, Russian spring-summer encephalitis virus, Semliki Forest virus, Sindbis virus (and variants Ockelbo and Babanki viruses), St. Louis encephalitis virus, Venezuelan equine encephalitis virus, West Nile virus, Western equine encephalitis virus, Yellow fever virus, Avian reticuloendotheliosis virus, Avian sarcoma and leukosis viruses, Border disease virus (sheep), Bovine immunodeficiency virus, Bovine leukemia virus, Bovine diarrhea virus, Caprine arthritis-encephalitis virus, Classical swine fever virus, Eastern equine encephalitis virus, Equine infectious anemia virus, Feline immunodeficiency virus, Feline leukemia virus, Feline sarcoma virus, Getah virus, Hog cholera virus, Japanese encephalitis virus, Lactic dehydrogenase-elevating virus (mice), Maedi/visna virus (sheep), Mouse hepatitis viruses, Mouse mammary tumor virus, Mucosal disease virus (cattle), Murine leukemia viruses (including Abelson, AKR, Friend, Maloney leukemia viruses, Progressive pneumonia virus of sheep, Rous sarcoma virus, Rauscher murine leukemia virus, Simian Immunodeficiency viruses (including African Green Monkey, Sooty mangabey, Stump-tailed macaque, pig-tailed macaque, Rhesus, Chimpanzee, and Mandrill viruses), Simian Type D retrovirus, Simian T-cell lymphotrophic viruses, Tick-borne encephalitis viruses (including European and far eastern tick-borne encephalitis viruses, Louping ill virus, and Powassan virus), Venezuelan equine encephalitis virus, Wesselsbron virus, and Western equine encephalitis virus, Woolly monkey sarcoma virus, berne virus, breda virus, infectious bronchitis virus (fowl), turkey bluecomb virus, transmissible gastroenteritis virus (swine), hemagglutinating encephalomyelitis virus (swine), porcine epidemic diarrhea virus, calf coronavirus, feline infectious peritonitis virus, feline enteric corona virus, canine coronavirus, mouse hepatitis viruses, rat coronavirus (sialodacryoadentis virus), rabbit coronavirus, bovine respiratory torovirus, porcine torovirus, feline torovirus, equine arteritis virus, lactate dehydrogenase-elevating virus (mice), simian hemorrhagic fever virus, Human metapneumovirus, Haemorrhagic septicaemia virus, Lelystad virus (porcine reproductive and respiratory syndrome virus), and VR2332 virus (swine), human respiratory coronavirus 229-E, human respiratory coronavirus OC43, human enteric corona virus, severe acute respiratory syndrome (SARS)-associated coronavirus and SARS Toronto Strain 2 virus.

Specific enveloped single-stranded negative sense RNA viruses which can be treated, prevented or managed by the methods of the present invention include, but are not limited to Alagoas virus, Bunyamwera virus, Bwamba virus, California encephalitis virus, Congo-Crimean hemorrhagic fever virus, Chandipura virus, Duvenhage virus, Guama virus, Guanarito virus, Hantaan virus, Influenza viruses A, B, and C, Isfahan virus, Jamestown Canyon virus, Junin virus (Argentine hemorrhagic fever virus), Lagos bat virus, La Crosse virus, Lassa virus, Lymphocytic choriomeningitis virus (LCM virus), Machupo virus, Maraba virus, Marburg virus, Measles virus, Mumps virus, Mokola virus, Muerto Canyon virus, Oriboca virus, Oropouche virus, Parainfluenza viruses 1 (Sendai virus), 2, 3, 4a, and 4b, Pichinde virus, Piry virus, Punto toro virus, Puumala virus, Rabies virus, Respiratory syncytial virus, Rift Valley fever virus, Sandfly fever-Naples virus, Sandfly fever-Sicilian virus, Seoul virus, Sin Nombre virus, Tacaribe virus, Tahyna virus, Tamiami virus, Vesicular stomatitis viruses (including New Jersey and Indiana strains), Akabane virus, Aino virus, Avian paramyxovirus 2 (Yucaipa virus), 3, 4, 5 (Kunitachi virus), 6, 7, 8, and 9, Bovine ephemeral fever virus, Bovine respiratory syncytial virus, Canine distemper virus, Dolphin and Porpoise distemper virus, Ebola virus (including subtypes Zaire, Sudan, and Reston), Equine morbillivirus, Infectious hematopoietic necrosis virus (fish), Influenza viruses of swine, horses, seals, and fowl, Kotonkan virus, Lymphocytic choriomeningitis virus , Marburg virus, Nairobi sheep disease virus, Newcastle disease virus (fowl), Obodhiang virus, Peste-des-petits-ruminants virus (sheep and goats), Pneumonia virus of mice, Pocine rubulavirus (la-Piedad-Michoacan-Mexico virus), Rabies virus, Rift Valley fever virus, Rinderpest virus, Simian parainfluenza virus 10, and Vesicular stomatitis viruses.

Specific double-stranded RNA viruses which can be treated, prevented or managed by the methods of the present invention include, but are not limited to Colorado tick fever virus, Reoviruses 1-3, Orungo virus, Kemerovo virus, Rotavirus groups A-F, Eyach virus, Ibaraki virus, Golden shiner virus, chub reovirus, African horsesickness viruses 1-9, Epizootic hemorrhagic disease viruses (deer), Infectious bursal disease virus (fowl), Infectious pancreatic necrosis virus (fish), Human rotaviruses, and Reoviruses 1-3.

In one embodiment, the invention encompasses the treatment, prevention or management of viruses that cause, lead to or are involved in cancer. Further, the invention encompasses the treatment, prevention or management of viral strains that are resistant to or exhibit resistance to conventional antiviral therapy.

In a specific embodiment of the invention, the virus to be treated is a herpes virus, or more specifically, the viruses to be treated are HSV-1 or HSV-2.

In another specific embodiment of the invention, the virus to be treated is not a herpes virus, or more specifically, the viruses to be treated are not HSV-1 or HSV-2. Further, in another alternative embodiment, the virus to be treated is not a retrovirus, or more specifically, the viruses to be treated are not HIV-1, HIV-2 or HTLV. Further, in another embodiment, the virus to be treated is not a hepatitis B virus, HCMV, MCMV, VZV, EBV, Measles virus, Punto Toro a, VEE, West Nile Virus, Vaccinia, Cow pox, Adenovirus Type 1, HPIV, Human metapneumoviurs, Haemorrhagic septicaemia virus, Parainfluenza type 3, Pichinde or rhinovirus.

In an alternative embodiment of the invention, the high dose methods for using sulfated polysaccharides of the invention can be used to treat, prevent or manage non-viral, microbial infections, including, but not limited to bacterial infections, parasitic infections and fungal infections. Bacterial infections that may be treated, prevented or managed by the methods of as described herein include both gram positive infections and gram negative infections. Specific bacterium and parasites that may be treated, prevented or managed by the methods as described herein include, but are not limited to, Chlamydia trachomatis; Helicobacter pylori; Lactobacilli; Plasmodium sp.; Escherichia coli; Staphylococcus aureus; Staphylococcus epidermis; Staphylococcus hemolyticus; Saccharomyces cerevisiae; Pseudomonas aeruginosa; Legionella pneumophila; Neisseria gonorrhea; Neisseria meningitidis; Plasmodium knowlesi; and Plasmodium falciparum.

The present invention provides methods for introducing a high dose of a sulfated polysaccharide or combination of such sulfated polysaccharides into the blood stream, lymphatic system, and/or extracellular spaces of the tissue of a patient in the treatment and/or prevention of viral infections, such as viral infections, bacterial infections or parasitic infections. The method comprises administering to a mammal at least sulfated polysaccharide that exhibits anti-viral activity in vitro, the sulfated polysaccharide having a sulfation which results in retention of anti-viral activity of the charged polysaccharide in vivo, e.g., sulfation that minimizes uptake by cells that have high charge density cell receptors.

Without being limited by theory, the Applicant believes that the sulfated polysaccharides of the invention have a high affinity for the lymph nodes thus have an increased activity against viruses which populate or gestate in the lymphatic system. Thus, the present invention encompasses a method of administering a sulfated polysaccharide of the invention directly to or targeted for the lymphatic system of a patient.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of viral infection. These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. In a particular embodiment, the methods of the present invention are particularly useful for patients at risk for the effects of bioterrorism, biomedical and biochemical weaponry including but not limited to military personnel and civilians in high risk target locations. Finally, the methods and compositions include the treatment of refractory patients or patients resistant to treatment such as resistance to reverse transcriptase inhibitors, protease inhibitors, etc.

5.1.1 Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lies preferably within a range of circulating concentrations that include the ED₅₀ with tolerable toxicity. The dosage may vary within this range depending upon the dosage form employed and the route of administration utilized. For any compound used in the method of the invention, the effective high dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are susceptible to infection with the virus to be treated, prevented, or managed (e.g. primary cells, transformed cell lines, patient tissue samples, etc.) or growth medium on which the virus to be treated, prevented, or managed can grow (e.g., LB broth/agar, YT broth/agar, blood agar, etc.) are exposed to or otherwise administered a compound of the invention and the effect of the compound upon the ability of the virus to grow is assessed. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a high dose of a sulfated polysaccharide of the invention or a pharmaceutically acceptable salt, solvate, hydrate, or stereoisomer thereof in the acute or chronic management of an infection or condition will vary with the nature and severity of the infection, and the route by which the active ingredient is administered. The high dose, and the dose frequency, will also vary according to the infection to be treated, the age, body weight, and response of the individual patient. Suitable dosing regimens can be readily selected by those skilled in the art with due consideration of such factors.

The high dose per day will vary according to the percent of sulfur with respect to the sugar residue wherein the more potent polysaccharides have a lower maximum tolerated dose and thus a lower high dose will be used. In a particular embodiment, the high dose administered will be within about 10% to 100% of the maximum tolerated dose of the particular polysaccharide administered, preferably within about 20% to about 95%; about 25% to about 95%; about 20% to about 90%; about 30% to about 90%; about 30% to about 85%; about 35% to about 85%; about 35% to about 80%; about 40% to about 80%; about 40% to about 75%; about 45% to about 75% of the maximum tolerated dose of the particular polysaccharide administered; most preferably within about 50% to about 75% of the maximum tolerated dose of the particular polysaccharide administered. Determination of the maximum tolerated dose may be done by assessing patient populations, e.g., establishing maximum tolerated dosages for a particular patient population. Determination of the maximum tolerated dose is also preferably determined patient-by-patient during the course of therapy. Maximum tolerated dose may be determined by titration of the polysaccharide against clinical parameters such as platelet count and blood anticoagulant activity, as well as the appearance of side effects listed elsewhere herein. For example, the maximum tolerated dose, in one embodiment, is that dose which causes a drop in blood platelet counts to below 40,000/mm³.

The high dose administered depends upon the specific compound to be used, and the weight and condition of the patient. The high dose within the scope of the present invention, includes in a separate and distinct embodiment, a daily dose in the range of from about 10 μg/kg to about 5000 mg/kg, about 20 μg/kg to about 2500 mg/kg, about 20 μg/kg to about 500 mg/kg, about 20 μg/kg to about 1500 mg/kg, about 20 μg/kg to about 400 mg/kg, about 30 μg/kg to about 200 mg/kg, preferably about 15 μg/kg to 150 mg/kg, preferably about 25 μg/kg to about 150 mg/kg, more preferably about 25 μg/kg to about 100 mg/kg; more preferably about 40 μg/kg to about 85 mg/kg, more preferably about 45 μg/kg to 85 mg/kg, and most preferably about 50 μg/kg to about 85 mg/kg, per day, depending on the particular polysaccharide administered.

The high dose within the scope of the present invention, includes in a separate and distinct embodiment, a daily dose of no more than 1 mg/kg, 2.5 mg/kg, 5 mg/kg, 10 mg/kg, 20 mg/kg, 30 mg/kg, 40 mg/kg, 50 mg/kg, 75 mg/kg, 100 mg/kg, 150 mg/kg, 200 mg/kg, 300 mg/kg, 400 mg/kg, 500 mg/kg, 750 mg/kg or 1000 mg/kg.

With respect to preferred sulfated polysaccharides, including, but not limited to commercial dextran sulfate, dextran sulfate having a percent of sulfur between about 2% and about 22%, sulfated polysaccharides having a percent of sulfur between about 6% and about 13%, periodate treated sulfated polysaccharides, and co-charged anionic sulfated polysaccharides, the high dose administered may include doses from about 10 μg/kg to 100 mg/kg, preferably 20 μg/kg to 90 mg/kg, and most preferably about 40 μg/kg to 85 mg/kg per day.

In general the therapeutically effective, short time interval for administration of a plurality of doses is the time sufficient to reduce viral load, inhibit viral replication, or otherwise achieve a recognizable therapeutic response while avoiding lethal or otherwise irrecoverable toxicity. In particular, the therapeutically effective, short time interval for administration is one to four times per day for 14 days, preferably once daily for 10 days, more preferably once daily for 7 days, and most preferably once daily for 4 days. In another preferred embodiment, the dose can be administered one to four times per day for 5 days, 4 days, 3 days, 2 days or 1 day. Furthermore, the methods of the invention can be repeated for a given patient, particularly for the treatment or management of chronic infections with the minimal interval of 2 days, 3 days, 4 days, 5 days, or 7 days. In general the therapeutically effective, short time interval for administration for an chronic infection may be one to four times per day every 3 days, every 5 days, every 7 days, every 14 days, every 21 days, every 30 days, every 60 days, every 90 days or once every 180 days. In a particular embodiment, the high dose is administered during the crisis period for an acute infection or an acute episode of a chronic infection. In general the administration during a crisis period will occur over a 10-14 day period but can vary with the severity of the crisis and the particular virus to be treated.

The time for administration of high doses of one or more of the polysaccharides of the invention is the time for which the dose or doses to achieve a measurable therapeutic benefit, e.g., reduction in viral load, reduction in viral replication, or an improvement in any other metric of viral infection or an improvement in patient health, for example, improved vital signs, reduced fever or other symptoms associated with viral infection. For example, administration for a “short time” encompasses administration for a time sufficient to cause a reduction in the level of viral nucleic acid in an infected tissue; reduction in a viral antigen in an infected tissue; a detectable reduction in viral replication over 1-7 days; measurable or noticeable improvement in any symptom of viral infection, whether quantifiable (e.g., body temperature, cytokine levels, levels of host cells bearing certain markers, etc.) or not (general feelings of malaise, level of energy). In various embodiments, therefore, a short time interval administration of a dose or doses of the polysaccharides of the invention is that time of administration that results in a reduction of viral nucleic acid, viral antigen, or the rate of viral replication of 10%, 15%, 20%, 25%, 30%, 35%, 40%, 45%, preferably 50%, 55%, 60%, 65%, 70%, more preferably 75%, 80%, 85%, even more preferably 90%, 95% or 99%, as compared to the pre-administration state. In other embodiments, a short time interval of administration of a dose or doses of the polysaccharides of the invention is that time of administration that results in the reduction of a physical symptom of a viral infection, or results in the change of a measure of the physical status of the infected individual measurably towards normal. In a specific embodiment, a short time interval of administration is that time required to reduce a fever by 0.5 degrees Fahrenheit, 1.0 degree, 1.5 degrees, 2.0 degrees, 2.5 degrees, 3.0 degrees, 3.5 degrees, 4.0 degrees, 4.5 degrees or 5.0 degrees Fahrenheit.

The time of administration of a series of high doses may be adjusted to avoid certain side effects. For example, in a preferred embodiment, the administration to an individual of one or more high doses is accompanied by testing the individual for platelet count; if platelet count drops below a predetermined level (e.g., 40,000/mm³; 45,000/mm³; 50,000/mm³; 55,000/mm³; 60,000/mm³; or 65,000/mm³) treatment is halted until platelet counts rise to within normal range.

In general, the high dose of a sulfated polysaccharide of the invention will be administered via bolus injection, intravenous injection, or oral delivery, preferably bolus injection. Furthermore, the administration time for a sulfated polysaccharide of the invention will vary with respect to the particular polysaccharide being administered, the size of the dose and the mode of administration. In general, the administration time for bolus injection will be the from about 1 second to about 10 minutes, from about 5 seconds to about 5 minutes, or from about 10 seconds to about 1 minute. The administration time for intravenous administration will be from about 5 minutes to about 4 hours; preferably from about 20 minutes to about 3 hours, more preferably about 30 minutes to about 2 hours and most preferably from about 45 minutes to about 1 hour.

Furthermore, the administration time of a high dose of a sulfated polysaccharide of the invention or a pharmaceutically acceptable salt, solvate, hydrate, or stereoisomer thereof in the acute or chronic management of an infection or condition will vary with the nature and severity of the infection, and the route by which the active ingredient is administered. In general, the high dose of a sulfated polysaccharide will be administered at an even rate over a 24 hour period, preferably a 12 hour period, more preferably a 4 hour period, most preferably a one hour period.

In another embodiment, the compounds of the invention can be administered as a predose in anticipation of potential infection or in a high dose (e.g., bolus) shortly after exposure to a virus or an infection.

For treatment of humans infected by viruses, the dose can per day is administered in about one to four divisions a day. Additionally, the recommended daily dose ran can be administered in cycles as single agents or in combination with other therapeutic agents. In one embodiment, the daily dose is administered in a single dose or in equally divided doses.

Different therapeutically effective high doses may be applicable for different infections, as will be readily determined by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

5.1.2 Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, β-interferons, alkylating agents, hormones or cytokines.

The sulfated polysaccharides of the invention can be administered or formulated in combination with antibiotics. For example, they can be formulated with a macrolide (e.g., tobramycin (Tobi®)), a cephalosporin (e.g., cephalexin (Keflex(®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cef mnetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The sulfated polysaccharides of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanola mine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The sulfated polysaccharides of the invention can be administered or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The sulfated polysaccharides of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

The sulfated polysaccharides of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and ninesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The sulfated polysaccharides of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, pleconaril, acyclovir, gacyclovir and cidofovir.

The sulfated polysaccharides of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114 (IDEC)) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The sulfated polysaccharides of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-alpha, and IFN-gamma).

The sulfated polysaccharides of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The sulfated polysaccharides of the invention can be administered or formulated in combination with β-interferons which include, but are not limited to, interferon beta-1a and interferon beta-1b.

The sulfated polysaccharides of the invention can be administered or formulated in combination with an absorption enhancer, particularly those which target the lymphatic system, including, but not limited to sodium glycocholate; sodium caprate; N-lauryl-ÿ-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancers can also be used. Thus, the invention also encompasses a pharmaceutical composition comprising one or more sulfated polysaccharides of the invention and one or more absorption enhancers.

The sulfated polysaccharides of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The compounds of the invention and the other therapeutics agent can act additively or, more preferably, synergistically. In a preferred embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent. In a separate embodiment, a compound of the invention is administered to a patient who has not previously undergone or is not currently undergoing treatment with another therapeutic agent, particularly an antiviral agent.

In one embodiment, the methods of the invention comprise the administration of one or more sulfated polysaccharides of the invention without an additional therapeutic agent. In a specific embodiment, the methods of the invention comprise the administration of one or more sulfated polysaccharides of the invention without a fibroblast growth inhibitor. In another specific embodiment, the additional therapeutic agent is administered as a high dose.

5.2 Periodate Treated and Co-Charged Anionic Polysaccharides

The invention encompasses sulfated polysaccharides that have been manipulated to reduce endocytosis by cell receptors and to increase the flexibility of the polysaccharide backbone to enable the efficient presentation of anionic charged groups to interact with regions on the targeted viruses.

One manipulation encompassed by the present invention is the treatment of sulfated polysaccharides with periodate. Periodate-treated anionic polysaccharides have increased flexibility due to periodate oxidation of some or all sugar residues. This treatment allows increased freedom of rotation and conformational flexibility of the polymers and provides flexible joints to facilitate biological interactions. Periodate-treated sulfated polysaccharides of the invention can have any counterion to ensure solubility including, but not limited to sodium, calcium, quaternary ammonium, and potassium.

Materials which may be periodate-treated and used within the methods and compositions described herein also include the polysaccharides of Table 1 below.

Other variations include the incorporation of non-sulfate groups, such as carboxymethyl groups and sulfonate groups. By lowering the degree of substitution of charge on the polysaccharide with either sulfonate or carboxymethyl groups, the ability of the polysaccharide to be endocyctosed by high charge receptors is greatly reduced, therefore increasing its plasma stability. Carboxymethyl dextran sulfate can be prepared using a modification of methods of preparation employed by others (McLaughlin and Hirbst, Can. J. Res. 28B; 731-736, 1950; Brown et al. Arkiv Kemi 22: 189-206 1964). Approximately 20 g of dextran is slurried in a mixture of isopropanol (350 ml) and 3.85M NaOH (40 ml) and is stirred for five minutes at 5° C. in a blender. Sodium chloroacetate (18 g) is added, and the whole mixture is stirred for 60 minutes at 5° C. under a nitrogen atmosphere, the mixture is removed from the blender and stored at 25° C. for three days. The degree of carboxymethyl substitution can be adjusted by varying the time at 25° C. from 1 day to 3 days as well as varying the mole ratio of CICH₂COONa to anhydroglucose from 1 to 4 and keeping the molar ratio of CICH₂COONa to NaOH to 1 to 1.4. After neutralization the sample is washed with 80% ethanol and dried.

In a preferred embodiment, the invention encompasses a method of treating or preventing a viral infection in a mammal which comprises administering to a mammal in need of such treatment or prevention a high dose of a co-charged anionic polysaccharide which has a percent of sulfation which enables maximal interaction with the virus and which is not substantially endocytosed or degraded by cell receptor binding in the mammal thereby retaining antiviral in vivo. In a particular embodiment, co-charged anionic polysaccharide is co-charged with carboxymethyl groups, sulfonate groups, sulfate groups or mixtures thereof; more preferably the co-charged anionic polysaccharide is co-charged with carboxymethyl groups. In a specific embodiment, the co-charged anionic polysaccharide is carboxymethyl dextran sulfate or carboxymethyl cellulose.

Listed in Table 1 below are examples of sulfated polysaccharides (not including dextran sulfate) that may be used in accordance with the high dosing methods described herein.

TABLE 1

| | |
|---|---|
| (14)-2-deoxy-2-sulfamido-3-O-sulfo-(14)-beta-D- | Periodate degraded heparin |
| glycopyranan (derivative of chitosan) | |
| 2-acetamido-2-deoxy-3-O-sulfo(14)-beta-D- | Peptidoglycan DS-4152 |
| glycopyranan (derivative of chitosan) | |
| Achranthese bidentata polysaccharide sulfate | Phosphorothioate oligodeoxynucleotides |
| Aurintricarboxylic acid | Polyacetal polysulfate |
| Calcium spirulan | Polyinosinic-polycytidylic acid |
| Carboxymethylchitin | Polysaccharides from Indocalamus tesselatus |
| | (bamboo leaves) |
| Chemically degraded heparin (Org 31733) | Prunellin |
| Chondroitin polysulfate | Rhamnan sulfate |
| Copolymer of sulphonic acid and biphenyl | Ribofuranan sulfate |
| disulphonic acid urea (MDL 10128) | |
| Curdlan sulfate | Sodium lauryl sulfate |
| Cyanovirin-N (from cyanobacterium) | Sulfate dodecyl laminarapentaoside (alkyl |
| | oligosaccharide) |
| Fucoidin | Sulfated bacterial glycosaminooglycan |
| Galactan sulfate | Sulfated dodecyl laminari-oligomer (alkyl |
| | oligosaccharide) |
| Glucosamine-6-sulfate (monosaccharide) | Sulfated gangliosides |
| Glycyrrhizin sulfate | Sulfated laminara-oligosaccharide glycosides |
| | synthesized from laminara-tetraose, laminara- |
| | pentaose, laminara-hexaose |
| Heparin | Sulfated N-deacetylatedchitin |
| Inositol hexasulfate | Sulfated octadecyl maltohexaoside (alkyl |
| | oligosaccharide) |
| Lentinan sulfate | Sulfated octadecyl ribofurnans |
| Mannan sulfate | Sulfated oligoxylan (heparin mimetic) |
| N-acylated heparin conjugates | Sulfated xylogalactans |
| N-carboxymethylchitosan-N,O-sulfate | Sulfatide (3 sulfogalactosylceramide) |
| Oligonucleotide-poly(L-lysine)-heparin complexes | Sulfoevernan |
| Pentosan polysulfate (xylanopolyhydrogen sulfate) | Xylomannan sulfate |

Each of sulfated polysaccharides listed above, as well as any other sulfated polysaccharide that has anti-viral activity in vitro, may be modified to bring their degree of sulfation or ionic charge to a level suitable for their use in the methods or compositions of the invention.

The invention further encompasses a method of treating or preventing a viral infection in a mammal which comprises administering a high dose of one or more compounds chosen from the group consisting of cellulose sulfate; (14)-2-deoxy-2-sulfamido-3-O-sulfo-(14)-beta-D-glycopyranan (derivative of chitosan); 2-acetamido-2-deoxy-3-O-sulfo(14)-beta-D-glycopyranan (derivative of chitosan); Achranthese bidentata polysaccharide sulfate; Aurintricarboxylic acid; Calcium spirulan; Carboxymethylchitin; Chemically degraded heparin (Org 31733); Chondroitin polysulfate; Copolymer of sulphonic acid and biphenyl disulphonic acid urea (MDL 10128); Curdlan sulfate; Cyanovirin-N (from cyanobacterium); Fucoidin; Galactan sulfate; Glucosamine-6-sulfate (monosaccharide); Glycyrrhizin sulfate; Heparin; Inositol hexasulfate; Lentinan sulfate; Mannan sulfate; N-acylated heparin conjugates; N-carboxymethylchitosan-N,O-sulfate; Oligonucleotide-poly(L-lysine)-heparin complexes; Pentosan polysulfate (xylanopolyhydrogen sulfate); Peptidoglycan DS-4152; Periodate degraded heparin; Phosphorothioate oligodeoxynucleotides; Polyacetal polysulfate; Polyinosinic-polycytidylic acid; Polysaccharides from Indocalamus tesselatus (bamboo leaves); Prunellin; Rhamnan sulfate; Ribofuranan sulfate; Sodium lauryl sulfate; Sulfate dodecyl laminarapentaoside (alkyl oligosaccharide); Sulfated bacterial glycosaminooglycan; Sulfated dodecyl laminari-oligomer (alkyl oligosaccharide); Sulfated gangliosides; Sulfated laminara-oligosaccharide glycosides synthesized from laminara-tetraose, laminara-pentaose, laminara-hexaose; Sulfated N-deacetylatedchitin; Sulfated octadecyl maltohexaoside (alkyl oligosaccharide); Sulfated octadecyl ribofurnans; Sulfated oligoxylan (heparin mimetic); Sulfated xylogalactans; Sulfatide (3 sulfogalactosylceramide); Sulfoevernan; and Xylomannan sulfate, wherein the percent of sulfation of said compound has been modified or controlled to enable maximal interaction of constituent sulfate groups with the virus causing the infection, and wherein the compound is not substantially endocytosed or degraded by cell receptor binding in the mammal, thereby retaining antimicrobial activity in vivo.

5.3 Pharmaceutical Compositions and Dosage Forms

Pharmaceutical compositions and single unit dosage forms comprising a high dose of sulfated polysaccharide of the invention, or a pharmaceutically acceptable salt, hydrate or stereoisomer thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Individual dosage forms are preferably suitable for bolus injection. Pharmaceutical compositions and dosage forms of the invention typically also comprise one or more pharmaceutically acceptable excipients. Sterile dosage forms are also contemplated.

In an alternative embodiment, pharmaceutical compositions encompassed by this embodiment include a high dose of a sulfated polysaccharide of the invention, or a pharmaceutically acceptable salt, hydrate or stereoisomer thereof, and at least one additional therapeutic agent. Examples of additional therapeutic agents include, but are not limited to, those listed above in section 5.1.2.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise sulfated polysaccharides of the invention, or a pharmaceutically acceptable salt, hydrate, or stereoisomers thereof comprise 0.1 mg to 1500 mg per unit to provide doses of about 10 μg/kg to 500 mg/kg per day and 100 μg/kg to 100 mg/kg per day for preferred polysaccharides of the invention.

5.3.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103 and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

5.3.2 Delayed Release Dosage Forms

Active ingredients of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

5.3.3 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions. Preferred modes of parenteral administration include intravenous administration and bolus injection, most preferably bolus injection.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

5.3.4 Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetainide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.3.5 Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). In a preferred embodiment of the invention, the sulfated polysaccharides of the invention have a molecular weight greater than about 500,000 when administered topically.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

5.3.6 Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The sulfated polysaccharides of the invention may also be administered directly to the lung by inhalation. For administration by inhalation, a sulfated polysaccharide can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a sulfated polysaccharide directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a sulfated polysaccharide to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a sulfated polysaccharide to the lung is a liquid spray device supplied, for example, by Aradigm Corporation. Liquid spray systems use extremely small nozzle holes to aerosolize liquid drug formulations that can then be directly inhaled into the lung.

In a preferred embodiment, a nebulizer device is used to deliver sulfated polysaccharides to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In a particularly preferred embodiment, an electrohydrodynamic (“EHD”) aerosol device is used to deliver sulfated polysaccharides to the lung. EHD aerosol devices use electrical energy to aerosolize liquid drug solutions or suspensions (see, e.g., Noakes et al., U.S. Pat. No. 4,765,539; Coffee, U.S. Pat. No., 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the sulfated polysaccharides formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of sulfated polysaccharides will be known to the skilled artisan and are within the scope of the invention.

Liquid drug formulations suitable for use with nebulizers and liquid spray devices and EHD aerosol devices will typically include a sulfated polysaccharide with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of sulfated polysaccharide. Preferably, this material is liquid such as an alcohol, glycol, polyglycol or a fatty acid. Other methods of formulating liquid drug solutions or suspension suitable for use in aerosol devices are known to those of skill in the art (see, e.g., Biesaiski, U.S. Pat. No. 5,112,598; Biesalski, U.S. Pat. No. 5,556,611, which are herein incorporated by reference) A sulfated polysaccharides can also be formulated in rectal or vaginal compositions such as suppositories or retention enemas, e.g., containing conventional suppository bases such as cocoa butter or other glycerides.

In addition to the formulations described previously, a sulfated polysaccharide can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver sulfated polysaccharides. Certain organic solvents such as dimethylsulfoxide can also be employed, although usually at the cost of greater toxicity. A sulfated polysaccharide can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macroinol. Sci. Rev. Macroinol. Chem., 1983, 23, 61; see also Levy et al., Science, 1985, 228, 190; During et al., Ann. Neurol., 1989, 25, 351; Howard et al., 1989, J. Neurosurg. 71, 105). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g. Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

5.3.7 Nutritional Products and Dietary Supplements

A high dose of the sulfated polysaccharides may be incorporated into nutritional products including, but not limited to food compositions, over the counter, and dietary supplements. The sulfated polysaccharides may be added to various foods so as to be consumed simultaneously. As a food additive, the sulfated polysaccharides of the invention may be used in the same manner as conventional food additives, and thus, only needs to be mixed with other components to enhance the taste. Taste enhancement includes, but is not limited to, imparting to food a refreshingness, vitality, cleanness, fineness, or bracingness to the inherent taste of the food.

It will be recognized that dietary supplements may not use the same formulation ingredients or have the same sterile and other FDA requirements as pharmaceutical compositions. The dietary supplements may be in liquid form, for example, solutions, syrups or suspensions, or may be in the form of a product for reconstitution with water or any other suitable liquid before use. Such liquid preparations may be prepared by conventional means such as a tea, health beverage, dietary shake, liquid concentrate, or liquid soluble tablet, capsule, pill, or powder such that the beverage may be prepared by dissolving the liquid soluble tablet, capsule, pill, or powder within a liquid and consuming the resulting beverage. Alternatively, the dietary supplements may take the form of tablets or capsules prepared by conventional means and optionally including other dietary supplements including vitamins, minerals, other herbal supplements, binding agents, fillers, lubricants, disintegrants, or wetting agents, as those discussed above. The tablets may be coated by methods well-known in the art. In a preferred embodiment, the dietary supplement may take the form of a capsule or powder to be dissolved in a liquid for oral consumption.

The amount of sulfated polysaccharides in a beverage or incorporated into a food product will depend on the kind of beverage, food and the desired effect. In general, a single serving comprises an amount of about 0.1% to about 50%, preferably of about 0.5% to about 20% of the food composition. More preferably a food product comprises sulfated polysaccharides in an amount of about 1% to about 10% by weight of the food composition.

Examples of food include, but are not limited to, confectionery such as sweets (candies, jellies, jams, etc.), gums, bean pastes, baked confectioneries or molded confectioneries (cookies, biscuits, etc.), steamed confectioneries, cacao or cacao products (chocolates and cocoa), frozen confectioneries (ice cream, ices, etc.), beverages (fruit juice, soft drinks, carbonated beverages), health drinks, health bars, and tea (green tea, black tea, etc.).

5.4 Assays and Animal Models

The sulfated polysaccharides, compositions and dosage forms of the invention can be tested in vitro or in vivo by a variety of methods known in the art to test antiviral activity. See, for example, the methods discussed below and used throughout the examples.

A number of assays may be employed in accordance with the present invention in order to determine the degree of anti-viral activity of a compound of the invention such as cell culture, animal models, and administration to human subjects. The assays described herein may be used to assay viral growth over time to determine the growth characteristics of a virus in the presence of a compound of the invention.

In one embodiment, a virus and a compound of the invention are added to a permissive cell line (e.g. primary cells, transformed cell lines, patient tissue samples, etc.) or growth medium (e.g., LB broth/agar, YT broth/agar, blood agar, etc.). The growth/infection of the virus can be compared the growth/infection of the virus in the absence of the compound of the invention. Anti-virus activity of the compound of the invention is demonstrated by a decrease in virus growth/infection in the presence of the compound of the invention. Any method known in the art can be used to determine the growth/infection including, but not limited to, immunofluorescent staining, immunoblot or detection of a virus-specific nucleic acid (e.g., by in situ hybridization, or after cell lysis by Southern blot or RT-PCR analysis), visual/microscopic inspection for cytopathic effect of growth/infection (e.g., cell rounding, cell detachment, cell lysis, formation of multinucleated syncytia), virus titer (e.g., plaque forming units, colony forming units, etc.), number of plaques/colonies. In a specific embodiment, the virus and the compound of the invention are added to the cells or growth medium at the same time. In another specific embodiment, the virus is added to the cells or growth medium before the compound of the invention. In another specific embodiment, the compound of the invention is added to the cells or growth medium before the virus.

In another embodiment, a virus and a compound of the invention are administered to animal subjects susceptible to infection with the virus. The incidence, severity, length, virus load, mortality rate of infection, etc. can be compared to the incidence, severity, length, virus load, mortality rate of infection, etc. observed when subjects are administered the virus alone (in the absence of a compound of the invention). Anti-virus activity of the compound of the invention is demonstrated by a decrease in incidence, severity, length, virus load, mortality rate of infection, etc. in the presence of the compound of the invention. In a specific embodiment, the virus and the compound of the invention are administered to the animal subject at the same time. In another specific embodiment, the virus is administered to the animal subject before the compound of the invention. In another specific embodiment, the compound of the invention is administered to the animal subject before the virus.

In another embodiment, the growth rate of the virus can be tested by sampling cell culture medium or biological fluids/clinical samples (e.g., nasal aspirate, throat swab, sputum, broncho-alveolar lavage, urine, saliva, blood, or serum) from human or animal subjects at multiple time points post-infection either in the presence or absence of a compound of the invention and measuring levels of virus. In specific embodiments, the growth rate of a virus is assayed by assessing the presence of virus in a sample after growth in cell culture, growth on a permissible growth medium, or growth in subject using any method well-known in the art, for example, but not limited to, immunoassay (e.g., ELISA; for discussion regarding ELISAs see, e.g., Ausubel et al., eds, 1994, Current Protocols in Molecular Biology, Vol. 1, John Wiley & Sons, Inc., New York at 11.2.1), immunofluorescent staining, or immunoblot analysis using an antibody which immunospecifically recognizes the virus to be assayed or detection of a virus-specific nucleic acid (e.g., by Southern blot or RT-PCR analysis, etc.).

In other specific embodiments, the growth rate of a virus is assayed after growth in a subject. Standard models of in vivo antiviral activity include, but are not limited to, a primo-infection cynomolgus monkey model (Le Grand et al., Symp. Nonhum Primate Models AIDS. 1993 Sep. 19-22, 11); and those described in The Handbook of Animal Models of Infection (Zak and Sande eds., Academic Press; 1st edition (1999), including but not limited to a Cytomegalovirus infections guinea pig model; a cytomegalovirus infection rat CMV model; a human cytomegalovirus infection of the SCID-hu (thy/liv) mouse model; an ocular cytomegalovirus infections in SCID-hu mice model; a simian varicella model; a varicella zoster infection of t-cells and skin in the SCID-hu mouse mode; a mouse model of influenza virus infection; a ferret model of influenza virus infection; a cotton rat model of respiratory syncytial virus; a transgenic mouse models for HBV infections; a duck model for hepatitis B infection; a woodchuck model of hepatitis B virus infection; adult mouse models for rotavirus; a macaques model of SIV infection; a SCID-hu thy-liv mouse models for HIV infection; and a chimpanzee model of HIV-1 infection.

In a specific embodiment, viral titers can be determined by obtaining cell culture medium or biological fluids/clinical samples from infected cells or an infected subject, preparing a serial dilution of the sample and infecting a monolayer of cells that are susceptible to infection with the virus (e.g. primary cells, transformed cell lines, patient tissue samples, etc.) at a dilution of the virus that allows for the emergence of single plaques. The plaques can then be counted and the viral titer expressed as plaque forming units per milliliter of sample.

In one specific embodiment, the growth rate of a virus in a subject can be estimated by the titer of antibodies against the virus in the subject. Antibody serum titer can be determined by any method well-known in the art, for example, but not limited to, the amount of antibody or antibody fragment in serum samples can be quantitated by, e.g., ELISA. Additionally, in vivo activity of a sulfated polysaccharide can be determined by directly administering the compound to a test animal, collecting biological fluids (e.g., nasal aspirate, throat swab, sputum, broncho-alveolar lavage, urine, saliva, blood, or serum) and testing the fluid for anti-virus activity.

In embodiments where samples to be assayed for virus levels are biological fluids/clinical samples (e.g., nasal aspirate, throat swab, sputum, broncho-alveolar lavage, urine, saliva, blood, or serum), the samples may or may not contain in tact cells. Samples from subjects containing intact cells can be directly processed, whereas isolates without intact cells may or may not be first cultured on a permissive cell line (e.g. primary cells, transformed cell lines, patient tissue samples, etc.) or growth medium (e.g., LB broth/agar, YT broth/agar, blood agar, etc.). Cell suspensions can be cleared by centrifugation at, e.g., 300×g for 5 minutes at room temperature, followed by a PBS, pH 7.4 (Ca⁺⁺ and Mg⁺⁺ free) wash under the same conditions. Cell pellets can be resuspended in a small volume of PBS for analysis. Primary clinical isolates containing intact cells can be mixed with PBS and centrifuged at 300×g for 5 minutes at room temperature. Mucus is removed from the interface with a sterile pipette tip and cell pellets can be washed once more with PBS under the same conditions. Pellets can then be resuspended in a small volume of PBS for analysis.

In another embodiment, a compound of the invention is administered to a human subject infected with a virus. The incidence, severity, length, viral load, mortality rate of infection, etc. can be compared to the incidence, severity, length, viral load, mortality rate of infection, etc. observed in human subjects infected with a virus in the absence of a compound of the invention or in the presence of a placebo. Anti-viral activity of the compound of the invention is demonstrated by a decrease in incidence, severity, length, viral load, mortality rate of infection, etc. in the presence of the compound of the invention. Any method known in the art can be used to determine anti-viral activity in a subject such as those described previously.

Additionally, in vivo activity of a sulfated polysaccharide can be determined by directly administering the compound to an animal or human subject, collecting biological fluids/clinical samples (e.g., nasal aspirate, throat swab, sputum, broncho-alveolar lavage, urine, saliva, blood, or serum) and testing the biological fluids/clinical samples for anti-viral activity (e.g., by addition to cells in culture in the presence of the virus).

In general, in vivo stability can be determined by a variety of models known to the skilled artisan. In particular, in vivo stability can be determined by a kidney perfusion assay. For either type of analysis, the test compound may be labeled, for example with tritium. A kidney perfusion technique is described in detail in Tay et al. (Am. J. Physiol., (1991), 260: F549-F554). Briefly, rat kidneys, e.g., from male Sprague-Dawley rats, are perfused with 5% bovine serum albumin (BSA) in modified Krebs Henseleit buffer containing amino acids and continually gassed with 95% O₂-5% CO₂. Samples that have been perfused may be subjected to ion-exchange chromatography using, for example, a 19×1/cm² column of sepharose Q. Samples are applied to the column in 6 M urea, 0.05 M Tris, 0.005% (w/v) Chaps, pH 7.0, and eluted with a linear gradient of 0.15-2.5 M NaCl in the same buffer at a flow rate of 0.5 ml/minute. Recoveries using this technique are very good.

The foregoing has demonstrated the pertinent and important features of the present invention. One of skill in the art will be appreciate that numerous modifications and embodiments may be devised. Therefore, it is intended that the appended claims cover all such modifications and embodiments.

### 6. WORKING EXAMPLES

The following examples are for the purpose of illustration only and are not intended as limiting the scope of the invention.

### 6.1 Example 1

### Synthesis of a Sulfated Dextran having a Sulfation of 9.5%

Dextran T20 (average molecular weight 20,000) was dried in vacuo at 60° C. overnight. The dried compound (100 g) was dissolved in 640 ml formamide (FA). Chlorosulfonic acid (CSA) 80 ml was added to FA 200 ml at a maximum of 45° C. in a 3-necked flask, then cooled in ice-water. The amount of CSA determines the ultimate sulfation of the sulfated dextran (180 ml CSA to 200 ml FA yields approximately 17% sulfur). The CSA/FA mix was slowly added (over two hours) to the dextran at a temperature of 40° C. After all of the CSA/FA was added, the mixture was stirred for 15 minutes at a temperature of 45° C. The mixture was cooled to 25° C. and 28% NaOH was added slowly to give a pH 7.5-8.5 with a maximum temperature of 50° C. For the first precipitation, 3 L of ethanol were added with stirring. Stirring was stopped and the mixture was allowed to stand. The supernatant was decanted and the precipitate was redissolved in 1.5 L of water. For the second precipitation 1.5 L ethanol were added with stifling and then the mixture was allowed to stand for two hours. The supernatant was decanted and the precipitate was redissolved in 900 ml of water, to which 17 g NaCl was added. For the third precipitation 800 ml ethanol were added with stirring and the mixture was allowed to stand for two hours. The optical rotation-maximum was measured. The supernatant was decanted and the precipitate was redissolved in 500 ml water. 2.8 g Na₂HPO₄ and 2.6 g NaH₂PO₄ were added. For the final precipitation 5 L ethanol were added and the precipitate was filtered on a glass filter and dried in vacuo at 50° C.

### 6.2 Example 2

### Periodate Oxidation

Following the modified method of Smith degradation used by Sandy J D, Biochem J., 177: 569-574, 1979; chrondroitin sulfate (240 mg) was dissolved in 0.25M NaClO₄ (47 ml) at room temperature. 5 ml of 0.5 M NaIO₄ was added and KOH was used to adjust the mixture to pH 5. The reaction was allowed to proceed in the dark for 72 hours. The mixture was then dialysed in visking tubing to remove the periodate.

### 6.3 Example 3

### Introduction of Anionic Sulfur Groups to Carboxymethyl Dextran

Sulfated Form of Carboxymethyl Dextran (Average mw 20,000) with a Sulfur Content of 9.5%.

Carboxymethyl dextran (CMD) is dried in vacuo at 60° C. overnight. CMD (100 g) is dissolved in 640 ml formamide (FA). Chlorosulfonic acid (CSA) 80 ml is added to FA 200 ml at maximum of 45° C. in a 3-necked flask then cooled in ice-water. The amount of CSA will determine the ultimate sulfur content of CMD (180 ml OSA to 200 ml FA yields approx 17% sulfur). The CSA/FA mix is added slowly (over 2 hours) to CMD at a temperature of 40° C. After all is added the mixture is stirred for 15 minutes at a temperature of 45° C. The mixture is cooled to 25° C. and 28% NaOH is added slowly to give a pH 7.5-8.5 with a maximum temperature of 50° C. For the first precipitation, 3 L of ethanol is added with stirring. Supernatant is decanted and then residue is redissolved in 1.5 L of water. For the second precipitation 1.5 L ethanol is added with stirring and then allowed to stand for 2 hours. Supernatant is decanted and residue is redissolved in 900 ml of water and then added to 17 g NaCl. For the third precipitation 800 ml ethanol is add with stirring and allowed to stand for 2 hours. The optical rotation maximum should be 0.3. Supernatant is decanted and the residue is redissolved in 500 m water. Add 2.8 g Na₂HPO₄ and 2.6 g NaH₂PO₄. For the final precipitation 5 L ethanol is added and filtered on a glass filter and is dried in vacuo at 50° C.

Sulfonated Form of Carboxymethyl Dextran (Average Molecular Weight 20,000).

Step 1. Dissolve 5 g dextran in water. Add 100 mg borohydride stir at room temp. for 30 min.

Step 2. Add sodium hydroxide pellets (10 g) and stir until dissolved and then sulfonate (12 g).

Step 3. Heat at 70° C. for 7 h. After 3 hours add a further 3 g of sulphonate. Continue heating for 4 hours.

Step 4. Neutralise with 5M HCl to pH 7.5 (Total volume(T)=75 ml) and gradually add 200 ml ethanol with good stirring. Stop stirrer and stand 1 hour.

Step 5. Decant supernatant; redissolve in water (T=60 ml) and add 150 ml ethanol with good stirring. Stand 1 hour.

Step 6. Repeat as Step 5.

Step 7. Decant off the supernatant—redissolve the residue in 60 ml water and ppte in 600 ml ethanol. Some concentrated sodium chloride solution may be added to the mixture to aid precipitation.

Step 8. Filter and dry in vacuo. Yield approx. 6 g.

### 6.4 Example 4

### In Vivo Anti-Viral Activity

The in vivo anti-viral activity of dextran sulfate and variants of sulfated dextrans are assessed in a pharmacokinetic study involving single intravenous doses of dextran sulfate (DS) given to three male and three female rats. Rats are Sprague-Dawley, previously cannulated in the vena cava. Blood is drawn at various times after injection and is assessed for anti-HIV activity in an acute infectivity cytoprotection assay system utilizing HIV-1 RF virus with CEN-SS cells using the MTS staining method for cell viability (based on Witvrouw et al., J. Acqur. Immun. Def. Syndr., 3:343-347, 1990).

### 6.5 Example 5

### Effect of High Dose on Pro-thrombin/Thrombin and Activated Partial Thromboplastin Time

The purpose of this study is to evaluate the effects of high dose dextran sulfate on prothrombin time (PT) and activated partial thromboplastin time (aPTT). All specimens are “spiked” with the test compound prior to submission to a Clinical Pathology Laboratory. The specimens are delivered along with reconstituted human plasma purchased from Sigma. Immediately prior to analysis 600 μl of the Sigma human plasma is added to each specimen.

A Bio-Merieux Coag-A-Mate MTX II Analyzer is used to measure Prothrombin Time (PT) and Activated Partial Thromboplastin Time (APTT). The PT reagent is Simplastin L and the APTT reagent is Platelin L; all reagents are obtained from Bio-Merieux. All specimens are run in duplicate. Coagulation control samples are analyzed immediately prior to testing.

| Parameter | Abbreviation | Units | Method |
|---|---|---|---|
| Prothrombin Time | PT | Seconds | Photo-optical |
| | | | hemostasis analyzer |
| Activated partial | APTT | Seconds | Photo-optical |
| Thromboplastin Time | | | hemostasis analyzer |

Specimen Disposition

The PT measuring time starts at five seconds and stopped at 60 seconds. The aPTT measuring time starts at five seconds and stops at 130 seconds.

Thrombocytopenia and Coagulation

Experiments to determine the effect of injected high dose dextran sulfate of various molecular weights on coagulation parameters are undertaken. Rats are given either varying high doses of each compound on consecutive days for ten days. On day 11, certain dosages are changed. At days 0, 5, 10 and 15 blood is drawn and assessed for aPTT and platelet counts.

Maximum Tolerated Dose

The maximum tolerated dose (MTD) of various sulfated polysaccharides is assessed in a series of experiments where groups of five rats are given varying high doses. Body weights and overall behavioral assessments are determined for five days after injection. Subsequently, rats are given a high dose injection and observed for a further five days. Finally, animals are given doses just below or at the MTD.

The MTD for dextran sulfate with a sulfur content of about 12.5% is about 500 mg/kg/day in dogs and about 300 mg/kg/day in monkeys. In rats the MTD of dextran sulfate of molecular weight of about 500,000 is about 800 mg/kg/day.

### 6.6 Example 6

### Biodistribution of a Compound of the Invention

Male Sprague-Dawley rats are obtained from Charles River Laboratories (Raleigh, N.C.; ca. 377-402 g) and are dosed with [³H]Des6 40K by intravenous bolus or oral gavage administration. Distribution of total tritium content in plasma, lymph, and cervical lymph nodes is quantitated in samples collected at 6 or 12 hours following dosing.

Rats are divided into three treatment administration groups. Doses are formulated in phosphate buffered saline vehicle (pH=7.4) so as to deliver them in approximate volumes of 1.8 mL/kg (iv) and 2.1 mL/kg (oral gavage).

Prior to the time of biological sample collection at 6 or 12 hours after dosing, animals are anesthetized with ketamine/xylazine (7:1, ca. 120 mg/kg), and the thoracic lymphatic duct is cannulated as described in Waynforth, H. B. and Flecknell, P. A. (1992). Experimental and Surgical Technique in the Rat, 2nd ed., Academic Press, New York. At the time of sample analysis, blood is collected by cardiac puncture and lymph was collected via the lymphatic duct cannula. Blood is processed for plasma by centrifugation at ca. 100 g for 10 minutes. Cervical lymph nodes are collected from each animal. Total radioactivity is quantitated in duplicate by liquid scintillation spectrometry for all biological samples collected.

### 6.7 Example 7

### Pox-Challenged Mice Studies

Evaluation of antiviral efficacy of DES6 was made in vivo employing Brighton cowpox strain challenged-female BALB/c mice (17-22 g). On Day 0 the mice received intratracheal administration of 50 μl DES6 (0.25 mg total). Approximately 2 h later the mice were challenged intranasally with 75 μl Cowpox (2.6×10⁵ PFU). On Day 10 the lungs of the mice were examined for viral load. For determination of virus titers, lungs were aseptically removed from mice, placed in labeled cryovials, and stored at −80° C. On the day of the assay, the lungs were thawed to room temperature in a biosafety cabinet and homogenized in 1 ml of tissue culture medium using disposable tissue grinders. Fluid from homogenized lungs was then subjected to ½ log dilutions on placed on Vero cells in a 96 well plate-based assay. After 4 days, the cells were stained with a neutral red solution and the absorbance at 540 nm obtained. The final dilution of lung homogenate fluid that gave an absorbance value of ˜80% of the cell control values were considered negative for virus replication. All samples were stored at −80° C. prior to processing, and all tissues were homogenized and tested in a single assay to ensure consistency among samples from different groups. Results with drug treated mice indicated that they had on average one log reduction in viral load as compared to control. Therefore with only one drug administration we were able to reduce the viral load in the lungs by a factor of 10 over 10 days or less.

### 6.8 Example 8

### Administration Regimens

Regimen 1

A human individual presents with a chronic viral infection determined to be caused by an RNA virus. The individual is treated by administration of 12.5 mg/kg dextran sulfate, 12.5% sulfur content, per day for fourteen days.

Regimen 2

A human individual presents with an acute viral infection determined to be caused by an RNA virus. The individual is treated by administration of 20 mg/kg dextran sulfate, 9.5% sulfur content, per day for four days.

Regimen 3

A human individual presents with an acute viral infection determined to be caused by a DNA virus. The individual is treated by administration of 20 mg/kg dextran sulfate, 9.5% sulfur content, per day for four days. During treatment, the patient is assessed daily for symptoms of toxicity, including hair loss, gastro-intestinal pain, bowel hemorrhaging, listlessness, thrombocytopenia, central nervous system damage, headache, pain, fever, asthenia, chills, malaise, syncope, vasodilatation, nausea, diarrhea, dyspepsia, anorexia, anemia, dizziness, muscle spasm, sinusitis, urticaria, alopecia, anorexia, constipation or anti-coagulation. At least once during treatment, the individual is assessed to determine if treatment has caused a drop in viral load, viral replication or viral DNA.

Regimen 4

A human individual presents with a chronic viral infection determined to be caused by an RNA virus. The individual is treated by oral administration of 10 mg/kg carboxymethyl dextran sulfate per day for four days.

Regimen 5

A human individual presents with an acute viral infection determined to be caused by a DNA virus. The individual is treated by intravenous administration of 20 mg/kg carboxymethyl cellulose sulfate per day for four days. During treatment, the patient is assessed daily for symptoms of toxicity listed in Regimen 3, above. At least once during treatment, the individual is assessed to determine if treatment has caused a drop in viral load, viral replication or viral DNA.

Regimen 6

A human individual presents with a chronic viral infection determined to be caused by a DNA virus. The individual is treated by intravenous administration of 10 mg/kg carboxymethyl cellulose sulfate per day for fourteen days. During treatment, the patient is assessed daily for symptoms of toxicity listed in Regimen 3, above. At least once during treatment, the individual is assessed to determine if treatment has caused a drop in viral load, viral replication or viral DNA.

Regimen 7

A nurse or other medical personnel is infected with a virus by a needle stick. The individual is administered a course of 8 mg/kg dextran sulfate (approximately 17% sulfur) per day for seven days, with monitoring for adverse side effects. The individual is subsequently monitored for 3-6 months for the appearance in the bloodstream of viral antigen or viral nucleic acid.

The foregoing has demonstrated the pertinent and important features of the present invention. One of skill in the art will be appreciate that numerous modifications and embodiments may be devised. Therefore, it is intended that the appended claims cover all such modifications and embodiments.

1. A method of treating a viral infection in a human comprising administering to a human in need thereof one or more doses of about 200 μg to 500 mg/kg/day of sulfated polysaccharide having a percent of sulfur above 2% and below 25%, for a time sufficient to reduce viral load, inhibit viral replication or otherwise achieve a detectable therapeutic response, wherein said time is no more than 14 consecutive days, and wherein said one or more doses cause only non-lethal or recoverable symptoms of toxicity.

2. The method of claim 1 wherein said symptoms of toxicity are hair loss, gastro-intestinal pain, bowel hemorrhaging, listlessness, thrombocytopenia, central nervous system damage, headache, pain, fever, asthenia, chills, malaise, syncope, vasodilatation, nausea, diarrhea, dyspepsia, anorexia, anemia, dizziness, muscle spasm, sinusitis, urticaria, alopecia, anorexia, constipation or anti-coagulation.

3. The method of claim 1 wherein the viral infection is an acute viral infection, a chronic viral infection or an acute episode of a chronic viral infection.

4. The method of claim 1 wherein said sulfated polysaccharide has a percent of sulfur above 6% and below 13%.

5. The method of claim 1 wherein the dose of said sulfated polysaccharide is 200 μg to 150 mg/kg/day.

6. The method of claim 1 wherein the dose of said sulfated polysaccharide is 250 μg to 100 mg/kg/day.

7. The method of claim 1 wherein the dose of said sulfated polysaccharide is 300 μg to 85 mg/kg/day.

8. The method of claim 1 wherein said plurality of doses are administered from one to four times daily for up to 14 days.

9. The method of claim 1 wherein said plurality of doses are administered from one to four times daily for 10 days.

10. The method of claim 1 wherein said plurality of doses are administered from one to four times daily for up to 7 days.

11. The method of claim 1 wherein said plurality of doses are administered from one to four times daily for 5 days.

12. The method of claim 1 wherein said plurality of doses are administered from one to four times daily for 4 days.

13. The method of claim 1 wherein said plurality of doses are administered from one to four times daily for 3 days.

14. The method of claim 1 wherein said plurality of doses are administered from one to four times daily for 2 days.

15. The method of claim 1 wherein said sulfated polysaccharide has a percent of sulfur above 6% and below 22%.

16. The method of claim 15 wherein the percent of sulfur is above 8%.

17. The method of claim 15 wherein the percent of sulfur is above 13%.

18. The method of claim 15 wherein the percent of sulfur is below 17%.

19. The method of claim 1 wherein the sulfated polysaccharide comprises D-glucopyranose residues linked by α-1,6 linkages.

20. The method of claim 1 wherein the sulfated polysaccharide comprises L-glucopyranose residues.

21.-38. (canceled)
